(19) United States
(12) Patent Application Publication
(10) Pub. No.: US 2024/0294693 A1
(43) Pub. Date: Sep. 5, 2024

(54) AROMATIC CARBODIIMIDES, PROCESS FOR THE PREPARATION AND USE THEREOF

(71) Applicant: LANXESS DEUTSCHLAND GMBH, COLOGNE (DE)

(72) Inventor: Wilhelm Laufer, Ellerstadt (DE)

(73) Assignee: LANXESS DEUTSCHLAND GMBH, COLOGNE (DE)

(21) Appl. No.: 18/578,370

(22) PCT Filed: Jul. 8, 2022
(86) PCT No.: PCT/EP2022/069107

§ 371 Date: Jan. 11, 2024

(30) Foreign Application Priority Data
Jul. 14, 2021 (EP) 21185586.1

(51) Int. Cl. C08G 18/02; C08G 18/09; C08G 18/79

(52) U.S. Cl. CPC C08G 18/025; C08G 18/095; C08G 18/794

(57) ABSTRACT

The invention relates to novel carbodiimides, to processes for the production thereof and to the use thereof as a stabilizer in ester-based polyols, in polyethylene terephthalate (PET), in polybutylene terephthalate (PBT), in polytrimethylene terephthalate (PTT), in copolyesters, in thermoplastic polyester elastomers (TPE E), in ethylene vinyl acetate (EVA), in polylactic acid (PLA) and/or in PLA derivatives, in polybutylene adipate-terephthalates (PBAT), in polybutylene succinates (PBS), in polyhydroxyalkanoates (PHA), in blends, in triglycerides, in thermoplastic polyurethanes, in polyurethane elastomers, in PU adhesives, in PU casting resins, for PU coatings or in PU foams.

Carbodiimides have proven advantageous in many applications, for example as hydrolysis inhibitors for thermoplastics, polyols, polyurethanes, triglycerides and lubricating oils.

For this purpose it is preferable to employ highly sterically hindered polycarbodiimides, though these are produced from very specific raw materials and are therefore very costly to procure. In addition, highly sterically hindered carbodiimides, for example those based on triisopropylphenyl isocyanate, have very high melting points, are insoluble, and can be introduced into the starting materials of the polyurethanes only with a considerable investment of time and equipment, if at all. Aromatic carbodiimides, which are based on cheaper raw materials, for example those described in EP 2997010 B1, can achieve very good hydrolysis inhibition in some ester-based polymers such as PET or PLA, but have the disadvantage of that they do not adequately inhibit hydrolysis in other applications, for example in polyurethane, and therefore cannot be employed universally. Carbodiimides of the prior art are often in a form that is difficult to meter, in particular in the form of a tacky composition.

There is therefore a need for novel carbodiimides which do not exhibit the disadvantages of the prior art, are simple to produce, exhibit high thermal stability, achieve excellent hydrolysis inhibition also in polyurethane applications and are additionally easier to meter.

This object was surprisingly achieved by carbodiimides of formula (I)

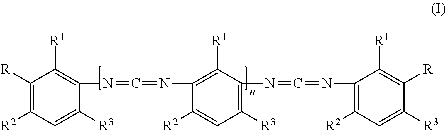

in which
R may be identical or different and is selected from —NCN—R¹ and —NHCOOR^{III}, wherein
R¹ represents C₁-C₂₂-alkyl, C₆-C₁₂-cycloalkyl, C₆-C₁₈-aryl or C₆-C₁₈-aralkyl, preferably triisopropylphenyl, and
R^{III} represents an alkylated polyoxyalkylene radical,
R¹, R² and R³ each independently of one another represent methyl, i-propyl or n-propyl, wherein on each benzene ring one of the radicals R¹, R² and R³ is methyl and
n is from 0 to 500, preferably n is from 1 to 50.

The molar mass of the alkylated polyoxyalkylene radical is preferably at least 200 g/mol, particularly preferably from 200-600 g/mol and most preferably from 350-550 g/mol.

The carbodiimide content (NCN content, measured by titration with oxalic acid) of the carbodiimides according to the invention is typically 2-17% by weight. To determine the NCN content, the NCN groups are reacted with oxalic acid added in excess and the unreacted oxalic acid is then potentiometrically back-titrated with sodium methoxide, taking into account the blank value of the system.

Preference is given to carbodiimides wherein R¹, R² and R³ each independently of one another represent methyl- or i-propyl-.

Preference is given to carbodiimides of formula (I) wherein R represents NCN—R¹, wherein n is from 0 to 500, preferably from 1 to 100 and most preferably from 1 to 50 and the carbodiimide content is preferably 10-17% by weight, particularly preferably 11-15% by weight and most preferably 13-14% by weight.

A further preferred embodiment relates to carbodiimides of formula (I) wherein R represents NHCOOR^{III}, wherein n is from 0 to 20, preferably from 1 to 10, particularly preferably from 3 to 8, and the carbodiimide content is preferably from 4% to 13% by weight, particularly preferably from 10% to 13% by weight.

Carbodiimides of formula (I) where R=—NCN—R¹, wherein R^{I} is as defined above and R¹, R² and R³ each independently represent methyl- or i-propyl-, wherein on each benzene ring one of the radicals R¹, R² and R³ is methyl, are solid and have softening points >40° C. They are therefore exceptionally suitable for stabilizing ester-based polymers, preferably polymers selected from polyester polyols, polyethylene terephthalate (PET), polybutylene terephthalate (PBT), polytrimethylene terephthalate (PTT), copolyesters such as modified polyesters of cyclohexanediol and terephthalic acid (PCTA), thermoplastic polyester elastomers (TPE E), ethylene vinyl acetate (EVA), polylactic acid (PLA) and/or PLA derivatives, polybutylene adipate-terephthalates (PBAT), polybutylene succinates (PBS), polyhydroxyalkanoates (PHA), polyurethane elastomers, preferably thermoplastic polyurethanes (TPU), and blends, preferably PA/PET or PHA/PLA blends.

The invention further relates to a process for stabilizing the ester-based polymers by addition of the above-mentioned carbodiimides. The above-mentioned carbodiimides are preferably added to the ester-based polymers using solids metering units.

Solids metering units in the context of the invention are preferably: single-, twin- and multi-screw extruders, continuous co-kneaders (Buss-type) and discontinuous kneaders, for example Banbury-type.

In a further embodiment of the invention, preference is given to carbodiimides of formula (I), wherein R represents-NHCOOR^{III} and R^{III} represents alkylated polyoxyalkylene and R¹, R² and R³ each independently of one another represent methyl- or i-propyl-, n is from 0 to 20, preferably n is from 1 to 10, particularly preferably from 1 to 4 and most preferably from 2 to 3 and the carbodiimide content is preferably from 2% to 10% by weight, particularly preferably 4% to 8% by weight and most preferably from 5% to 7% by weight.

Preferred alkylated polyoxyalkylene radicals are monoalkylated polyethylene glycol ethers, particularly preferably polyethylene glycol monomethyl ethers, in particular those having molar masses of 200-600 g/mol, preferably of 350-550 g/mol.

The above-mentioned carbodiimides of formula (I) where R=—NHCOOR^{III}, wherein R^{III} is an alkylated polyoxyalkylene radical, are typically liquid at room temperature, thus also allowing—in contrast to most solid carbodiimides—incorporation into liquid polyester polyols as used for the production of TPU and PU foams.

The invention therefore also relates to a process for stabilizing TPU and PU foams, wherein the above-mentioned carbodiimides are added to the liquid polyester polyols from which the TPU and PU foams are produced.

The invention further relates to a process for stabilizing ester-based oils and/or lubricants or greases, wherein the above-mentioned carbodiimides are added to the ester-based polymers.

The concentration of the carbodiimides of formula (I) according to the invention in ester-based polymers/in ester-based oils, lubricants or greases is preferably from 0.1% to 5% by weight, preferably from 0.5% to 3% by weight, particularly preferably from 1% to 2% by weight.

The carbodiimides according to the invention preferably have average molar masses (Mw) of 1000 to 20 000 g/mol, preferably of 1500 to 5000 g/mol, particularly preferably of 2000 to 4000 g/mol.

Preference is also given to carbodiimides having a polydispersity D=Mw/Mn of 1.2 to 2, particularly preferably of 1.4 to 1.8.

The scope of the invention encompasses all hereinabove- and hereinbelow-recited general definitions of radicals or definitions given in preferred ranges, indices, parameters, and elucidations among themselves, i.e. including between the respective ranges and preferred ranges in any desired combination.

The present invention further provides for producing the carbodiimides according to the invention by carbodiimidization of aromatic diisocyanates of formula (II)

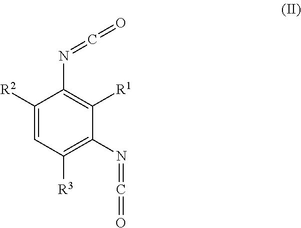

to eliminate carbon dioxide at temperatures of 80° C. to 200° C. in the presence of catalysts and optionally solvent and subsequently functionalizing the free NCO groups with alcohols of formula NOR^{III}, wherein R¹ to R³ and R^{I} to R^{III} are as defined for the compounds of formula (I).

In this process, the aromatic diisocyanates of formula (II) employed are preferably aromatic diisocyanates of formulae (III) and/or (IV):

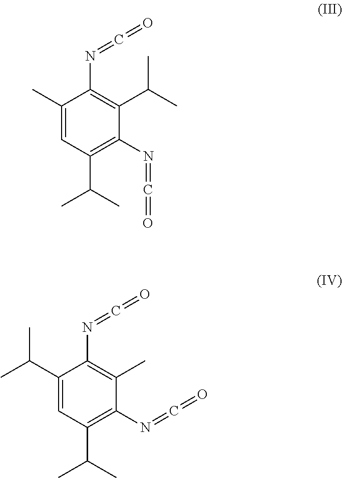

It is particularly preferable to employ mixtures of the diisocyanates of formulae (III) and (IV), preferably in the ratio 60:40 to 95:5, particularly preferably 70:30 to 90:10.

The aromatic diamines required for the production of the diisocyanates may—as is known to those skilled in the art—be produced by a Friedel—Crafts alkylation of the corresponding tolylenediamines with the corresponding alkene or haloalkane. These diamines are subsequently reacted with phosgene to afford the corresponding diisocyanate.

To produce the carbodiimides according to the invention, the diisocyanates, for example of formula (III) and/or (IV), may advantageously be condensed in the presence of catalysts and optionally a further monoisocyanate of the group R^{I} to eliminate carbon dioxide at elevated temperatures, preferably temperatures of 80-200° C., particularly preferably of 100° C. to 180° C., very particularly preferably of 120-140° C. Processes suitable therefor are described for example in DE-A 1130594 and DE-A 11564021.

In one embodiment of the invention, phosphorus compounds are preferred as catalysts for the production of the compounds of formula (I). Phosphorus compounds used are preferably phospholene oxides, phospholidenes or phospholine oxides and the corresponding phospholene sulfides. Further catalysts that may be used are tertiary amines, basic metal compounds, alkali metal and alkaline earth metal oxides, hydroxides, alkoxides or phenoxides, metal carboxylate salts and non-basic organometallic compounds.

The carbodiimidization can be conducted either in substance or in a solvent. Preferably employed solvents are alkylbenzenes, paraffin oils, polyethylene glycol dimethyl ethers, ketones or lactones.

In one embodiment of the present invention, the temperature of the reaction mixture is to this end reduced to 50-120° C., preferably 60-100° C., particularly preferably to 80-90° C. and the catalysts are distilled off at reduced pressure. In a preferred production variant of the carbodiimides according to the invention, the excess diisocyanate is subsequently distilled off at temperatures of 150-200° C., preferably 160-180° C. Subsequently, the free terminal isocyanate groups of the carbodiimides are reacted with alcohols, preferably in a slight excess of —OH groups, optionally in the presence of a PU catalyst known to those skilled in the art, preferably tertiary amines or organotin compounds, particularly preferably DBTL (dibutyltin dilaurate) or DOTL (dioctyltin dilaurate). The amount of substance ratio (molar ratio) of alcohols to carbodiimides is preferably 1.005-1.05:1, particularly preferably 1.01-1.03:1, based on the N═C═O groups present.

In a further embodiment of the present invention, to interrupt the carbodiimidization the temperature of the reaction mixture is reduced to a value in the range from 50° C. to 120° C., preferably from 60° C. to 100° C., particularly preferably to from 80° C. to 90° C., and optionally after addition of a solvent, preferably selected from the group of alkylbenzenes, particularly preferably toluene, the free terminal isocyanate groups of the carbodiimides are reacted with alcohols, preferably in a slight excess of —OH groups, optionally in the presence of a PU catalyst known to those skilled the art, preferably tertiary amines or organotin compounds, particularly preferably DBTL (dibutyltin dilaurate) or DOTL (dioctyltin dilaurate). The amount of substance ratio of alcohols to carbodiimides is preferably 1.005 to 1.05:1, particularly preferably 1.01 to 1.03:1, based on the N═C═O groups present.

After complete reaction, the catalyst, and optionally the solvent, is preferably distilled off at temperatures of 80-200° C. at reduced pressure.

The present invention additionally provides a further process for producing the carbodiimides according to the invention by a partial, preferably <50%, functionalization of the free NCO groups of aromatic diisocyanates of formula (II)

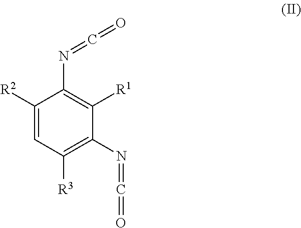

with alcohols of formula HOR^{III} and subsequent carbodiimidization to eliminate carbon dioxide at temperatures of 80° C. to 200° C. in the presence of catalysts and optionally solvent, wherein R¹ to R³ and R^{III} are as defined for the compounds of formula (I).

In this process too, the aromatic diisocyanates of formula (II) employed are preferably aromatic diisocyanates of formula (III):

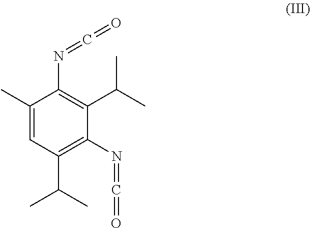 [SMILES: Cc1cc(C(C)C)c(N=C=O)c(C(C)C)c1N=C=O]

and/or of formula (IV)

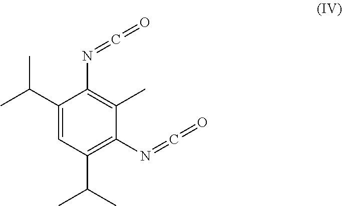 [SMILES: Cc1c(N=C=O)c(C(C)C)cc(C(C)C)c1N=C=O]

The carbodiimides according to the invention are preferably purified after production thereof. The crude products may be purified by distillation and/or by solvent extraction. Suitable solvents for the purification which may be employed with preference are polyethylene glycol dimethyl ethers, alkylbenzenes, paraffin oils, alcohols, ketones or esters. These are commercially available solvents.

The present invention further provides for producing the carbodiimides according to the invention by carbodiimidization of aromatic diisocyanates of formula (II)

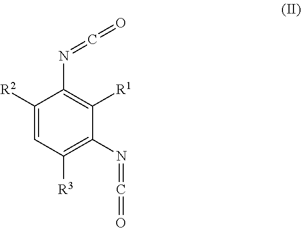

to eliminate carbon dioxide at temperatures of 80° C. to 200° C. in the presence of catalysts and optionally solvent, wherein before, during or after the carbodiimidization of the diisocyanates, monoisocyanates of formula OCN—R^{I} are added and wherein R¹ to R³ and R^{I} are as defined for the compounds of formula (I). In the case of addition after carbodiimidization of the diisocyanates, the carbodiimidization is continued to convert remaining isocyanate (end) groups of carbodiimides into groups of formula-NCN—R^{I} with monoisocyanate. In the case of addition of the monoisocyanates before or during the carbodiimidization of the diisocyanates, the functionalization of the end groups occurs automatically during the carbodiimidization.

In this process too, the aromatic diisocyanates of formula (II) employed are preferably aromatic diisocyanates of formula (III):

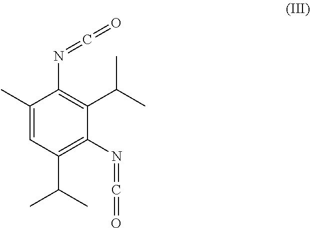 [SMILES: Cc1cc(C(C)C)c(N=C=O)c(C(C)C)c1N=C=O]

and/or of formula (IV)

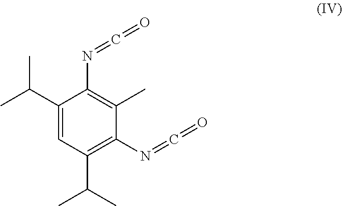 [SMILES: Cc1c(N=C=O)c(C(C)C)cc(C(C)C)c1N=C=O]

The monoisocyanate employed is preferably triisopropylphenyl isocyanate.

The present invention further provides a composition comprising
at least one ester-based polymer and
at least one inventive carbodiimide of formula (I).

The ester-based polymers are preferably polymers selected from polyester polyols, polyethylene terephthalate (PET), polybutylene terephthalate (PBT), polytrimethylene terephthalate (PTT), copolyesters such as modified polyesters of cyclohexanediol and terephthalic acid (PCTA), thermoplastic polyester elastomers (TPE E), ethylene vinyl acetate (EVA), polylactic acid (PLA) and/or PLA derivatives, polybutylene adipate-terephthalates (PBAT), polybutylene succinates (PBS), polyhydroxyalkanoates (PHA), polyurethane elastomers, preferably thermoplastic polyurethanes (TPU), and blends, preferably PA/PET or PHA/PLA blends.

In a particularly preferred embodiment of the invention, the ester-group-containing polymers are thermoplastic polyurethanes (TPU).

The concentration of inventive carbodiimides of formula (I) in the composition according to the invention is preferably 0.1-5% by weight, preferably 0.5-3% by weight, particularly preferably 1-2% by weight.

The polyester polyols as ester-based polymers are preferably long-chain compounds preferably having a molecular weight (in g/mol) of up to 2000, preferably between 500-2000 and particularly preferably between 500-1000.

The term “polyester polyols” in the context of the invention encompasses both long-chain diols and triols, and also compounds having more than three hydroxyl groups per molecule.

It is advantageous when the polyester polyol has an OH number of up to 200, preferably between 20 and 150 and particularly preferably between 50 and 115. Polyester polyols that are reaction products of different polyols with aromatic or aliphatic dicarboxylic acids and/or polymers of lactones are especially suitable.

The polyester polyols employed in the context of the invention are commercially available compounds obtainable from Covestro Deutschland AG under the trade names Baycoll® and Desmophen®.

The present invention further provides a process for producing the inventive carbodiimides of formula (I) where R=—NCN—R^{I}, wherein after the carbodiimidization the melt is pelletized, preferably on pelletizing lines, in unpurified form or after purification. Both customary pelletizing systems and customary granulating systems may be employed. These are obtainable for example from Sandvik Holding GmbH or GMF Gouda.

The inventive carbodiimides of formula (I) where R=—NCN—R^{I} and R^{I}=triisopropylphenyl are very particularly suitable.

The present invention additionally relates to the use of the carbodiimides according to the invention as inhibitors against hydrolytic decomposition in ester-based polymers, preferably polymers selected from polyester polyols, polyethylene terephthalate (PET), polybutylene terephthalate (PBT), polytrimethylene terephthalate (PTT), copolyesters such as modified polyesters of cyclohexanediol and terephthalic acid (PCTA), thermoplastic polyester elastomers (TPE E), ethylene vinyl acetate (EVA), polylactic acid (PLA) and/or PLA derivatives, polybutylene adipate-terephthalates (PBAT), polybutylene succinates (PBS), polyhydroxyalkanoates (PHA), polyurethane elastomers, preferably thermoplastic polyurethanes (TPU), and blends, such as preferably PA/PET or PHA/PLA blends, or in triglycerides, preferably trimethylolpropane trioleate (TMP oleate), in oil formulations for the lubricant industry, in PU adhesives, in PU casting resins. The use in urethanes comprises inter alia PU foams, PU coatings for wood, leather, synthetic leather and textiles. Use in thermoplastic polyurethanes (TPU) is particularly preferred.

The examples which follow serve to elucidate the invention without any limiting effect.

### WORKING EXAMPLES

Tests were carried out on:
1) CDI A: a solid carbodiimide having an NCN content of about 12% by weight based on about 80% by weight of diethyltolylene-2,4-diisocyanate and 20% by weight of diethyltolylene-2,6-diisocyanate end-functionalized with cyclohexanol (comparative example analogous to EP 2997010 B1).
2) CDI (B): a high-viscosity carbodiimide having an NCN content of about 14% by weight based on 80% by weight of diethyltolylene-2,4-diisocyanate and 20% by weight of diethyltolylene-2,6-diisocyanate end-functionalized with —NCN—R^{I}, wherein R^{I}=triisopropylphenyl and n>40 (comparative example).
3) CDI (C): a solid polymeric carbodiimide having an NCN content of about 14% by weight based on diisopropyltolylene diisocyanate (formulae III and IV in a weight ratio of about 1:4), end-functionalized with —NCN—R¹, wherein R^{I}=triisopropylphenyl and n>40 (inventive example).
4) CDI (D): a solid polymeric carbodiimide based on diisopropyltolylene diisocyanate, end-functionalized with ethylamine (comparative example analogous to CN 105778026).
5) CDI (E): a solid polymeric carbodiimide having an NCN content of about 6-7% by weight based on diisopropyltolylene diisocyanate (formulae III and IV in a weight ratio of about 1:4), end-functionalized with methyl polyethylene glycol (Mw about 550 g/mol), wherein n=4-5 (inventive example).

### Ester-Based Polymers:

6) Unstabilized thermoplastic polyurethane (TPU) obtainable from Covestro AG under the name Desmopan.

### Production of Carbodiimide CDI (A)

A baked-out and nitrogen-filled 250 ml four-necked flask was initially charged with 150 g of diisocyanates and 37.5 g of cyclohexanol under a nitrogen stream. 50 mg of 1-methylphospholene oxide were added and the mixture was then heated slowly to 180° C. Carbodiimidization was then carried out at 180° C. until an NCO content of <1% by weight had been achieved.

### Production of Carbodiimides CDI (B) and CDI (C):

A baked-out and nitrogen-filled 250 ml four-necked flask was initially charged with 150 g of diisocyanates and 37.5 g of triisopropylphenyl isocyanate under a nitrogen stream. 50 mg of 1-methylphospholene oxide were added and the mixture was then heated slowly to 180° C. Carbodiimidization was then carried out at 180° C. until an NCO content of <1% by weight had been achieved.

### Production of Carbodiimide CDI (D)

A baked-out and nitrogen-filled 250 ml four-necked flask was initially charged with 150 g of diisocyanates and 7.0 g of ethylamine under a nitrogen stream. 50 mg of 1-methylphospholene oxide were added and the mixture was then heated slowly to 180° C. Carbodiimidization was then carried out at 180° C. until an NCO content of <0.1% by weight had been achieved.

### Production of Carbodiimide CDI (E)

A baked-out and nitrogen-filled 250 ml four-necked flask was initially charged with 150 g of diisocyanates and 100 g of MPEG (methyl polyethylene glycol, Mw of about 550 g/mol). 50 mg of 1-methylphospholene oxide were added and the mixture was then heated slowly to 180° C. Carbodiimidization was then carried out at 180° C. until an NCO content of <0.1% by weight had been achieved.

### Hydrolysis Inhibition in Thermoplastic Polyurethane (TPU)

To evaluate the hydrolysis inhibition in TPU, 1.5% by weight respectively of the carbodiimides investigated were dispersed into TPU using a ZSK 25 laboratory twin screw extruder from Werner & Pfleiderer prior to the measurement described below. The standard test specimens used for measuring breaking strength were then produced from the resulting pellets on an Arburg Allrounder 320 S 150-500 injection-moulding machine.

For the hydrolysis test, these standard test specimens were stored in water at a temperature of 80° C. and their breaking strength in MPa was measured.

The results are shown in table 1:

TABLE 1

| Breaking | Ex. 1 | Ex. 2 | Ex. 3 | Ex. 4 |
|---|---|---|---|---|
| strength | (comp.) | (comp.) | (comp.) | (inv.) |
| (MPa) | (TPU) | (TPU, CDI A) | (TPU/CDI D) | (TPU/CDI E) |
| 0 days | 30 | 30 | 31 | 32 |
| 5 days | 28 | 30 | 30 | 32 |
| 10 days | 26 | 30 | 30 | 31 |
| 15 days | 12 | 28 | 30 | 30 |
| 20 days | 6 | 25 | 30 | 30 |
| 30 days | 0 | 5 | 28 | 30 |
| 80 days | — | — | 18 | 30 |
| 85 days | — | — | 5 | 29 |
| 90 days | — | — | | 28 |

comp. = comparative example,
inv. = inventive

The results from table 1 show that the inventive carbodiimides achieve markedly better hydrolysis inhibition relative to the prior art.

### Solubility in Polyester Polyol

The stabilization of the ester-based TPU elastomers to hydrolysis is in principle carried out directly during production. To this end, the carbodiimide is normally added to the polyester polyol or polyester plasticizer before the reaction with isocyanates to afford the polyurethane is carried out. The solubility of the carbodiimide is therefore important. Table 2 shows the different carbodiimides in a standard polyester polyol based on adipic acid and ethanediol and having a Mw=2000 (Desmophen 2000 MM from Covestro AG) at 80° C.

TABLE 2

| | Ex. 5 (comp.) | Ex. 6 (inv.) |
|---|---|---|
| | (CDI D) | (CDI E) |
| Solubility in | insoluble | soluble |
| polyester polyol | | |

### Hydrolysis Inhibition in Polyethylene Terephthalate (PET)

To evaluate the hydrolysis inhibition in PET, 1.5% by weight respectively of the carbodiimides investigated were dispersed into PET using a ZSK 25 laboratory twin screw extruder from Werner & Pfleiderer prior to the measurement described below. The F3 standard test specimens for measurement of breaking strength were then produced from the resultant pellets in an Arburg Allrounder 320 S 150-500 injection-moulding machine.

For the hydrolysis test, these F3 standard test specimens were stored in water at a temperature of 90° C. and the breaking strength thereof was measured in MPa. Table 2 shows the relative breaking strengths=(breaking strength after x days of storage/breaking strength after 0 days)×100. The lower limit for relative breaking strength is usually 70-75%.

The results are shown in table 3:

TABLE 3

| Relative breaking | Ex. 7 (comp.) | Ex. 8 (comp.) | Ex. 9 (inv.) |
|---|---|---|---|
| strength (%) | (PET) | (PET/CDI A) | (PET/CDI C) |
| 0 days | 100 | 100 | 100 |
| 5 days | 100 | 100 | 100 |
| 10 days | 51 | 100 | 100 |
| 15 days | — | 100 | 100 |
| 20 days | — | 100 | 100 |
| 25 days | — | 52 | 81 |
| 28 days | — | — | 41 |

comp. = comparative example,
inv. = inventive

### Tests on Pelletizability and Meterability of the Solid Carbodiimides

For clarification of the processability, handling and meterability of the different solid carbodiimides, these were compared in terms of appearance, pelletizability and softening point. The softening points were determined using a Koffler bench.

The results are shown in table 4:

| | | | meterability | Softening |
|---|---|---|---|---|
| | Appearance | pelletiz- | (T up to | point |
| Carbodiimide | (at RT) | ability | 40° C.) | (° C.) |
| CDI (B), | soft, tacky | not possible | — | <20 |
| comp. | composition | | | |
| CDI (D), | hard, tacky | not possible | — | >120 |
| comp. | composition | | | |
| CDI (C), | solid, brittle | very good | very good | about 80 |
| required | | | | |

comp. = comparative example,
inv. = inventive

The results of table 4 show that the inventive carbodiimides based on diisopropyl, tolylene end-capped with a monoisocyanate show, compared to the polymeric carbodiimide based on diethyltolylene diisocyanate also end-capped with a monoisocyanate and compared to carbodiimide D, exceptional pelletizability and a high softening point, thus entailing advantages in the processing and metering of the solid in the stabilization of the ester-based polymers. The carbodiimide E according to the invention is liquid and may be added as such.

1. A carbodiimide of formula (I) 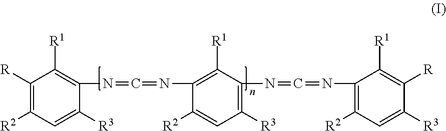
in which
R may be identical or different and is selected from —NCN—R^{I}— and —NHCOOR^{III}, wherein
R^{I} represents C₁-C₂₂-alkyl, C₆-C₁₂-cycloalkyl, C₆-C₁₈-aryl or C₆-C₁₈-aralkyl, and
R^{III} represents an alkylated polyoxyalkylene radical,
R¹, R² and R³ each independently of one another represent methyl, i-propyl or n-propyl, wherein on each benzene ring one of the radicals R¹, R² and R³ is methyl and
n is from 0 to 500.

2. The carbodiimide according to claim 1, wherein R¹, R² and R³ each independently of one another represent methyl- or i-propyl-.

3. The carbodiimide according to claim 1, wherein carbodiimide content is 2-17% by weight.

4. The carbodiimide according to claim 1, wherein in formula (I) R represents NCN—R^{I}, and carbodiimide content is 10-17% by weight.

5. The carbodiimide according to claim 1, wherein in formula (I) R represents —NHCOOR^{III}, n is from 0 to 20, and carbodiimide content is from 4% to 13% by weight.

6. The carbodiimide according to claim 1, wherein in formula (I) R represents —NHCOOR^{III} and R^{III} represents an alkylated polyoxyalkylene radical, n is from 0 to 20, and carbodiimide content is from 2-10% by weight.

7. The carbodiimide according to claim 6, wherein R^{III} represents monoalkylated polyethylene glycol ethers having molar masses of 200-600 g/mol.

8. A process for producing carbodiimides according to claim 1, comprising the steps of:
a) carbodiimidization of aromatic diisocyanates of formula (II) 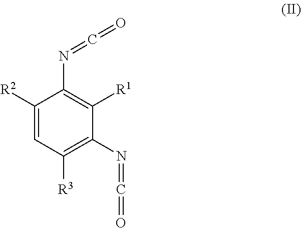
to eliminate carbon dioxide at temperatures of 80° C. to 200° C. in the presence of catalysts and optionally solvent and
b) functionalization of the free NCO groups of the carbodiimides obtained in step a) with alcohols of formula NOR^{III}, wherein R¹ to R³ and R^{III} are as defined for the compounds of formula (I).

9. A process for producing carbodiimides according to claim 1, comprising the steps of:
a) partial functionalization of the free NCO groups of aromatic diisocyanates of formula (II) 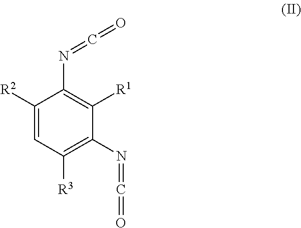
with alcohols of formula NOR″ and
b) subsequent carbodiimidization of the partially functionalized aromatic diisocyanates of formula (II) obtained in step a) to eliminate carbon dioxide at temperatures of 80° C. to 200° C. in the presence of catalysts and optionally solvent,
wherein R¹ to R³ and R^{III} are as defined for the compounds of formula (I).

10. A process for producing carbodiimides according to claim 1, comprising carbodiimidization of aromatic diisocyanates of formula (II) 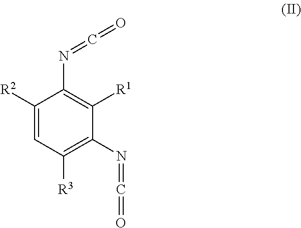
to eliminate carbon dioxide at temperatures of 80° C. to 200° C. in the presence of catalysts and optionally solvent, wherein before, during or after the carbodiimidization of the diisocyanates, monoisocyanates of formula OCN—R^{I} are added and wherein R¹ to R³ and R^{I} are as defined for the compounds of formula (I).

11. The process according to claim 8, wherein the aromatic diisocyanates of formula (II) employed are compounds of formulae (III) 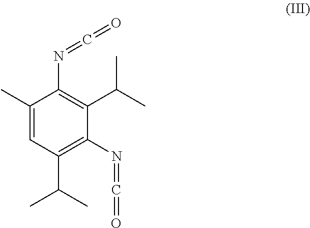 [SMILES: Cc1cc(C(C)C)c(N=C=O)c(C(C)C)c1N=C=O]
and/or (IV)

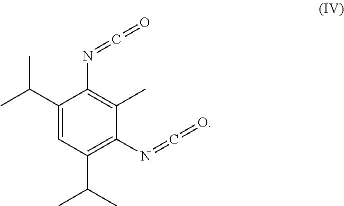 [SMILES: Cc1c(N=C=O)c(C(C)C)cc(C(C)C)c1N=C=O]

12. The process according to claim 8, wherein R=—NCN—R^{I}, wherein R^{I} is as defined for the compounds of formula (I) and the melt of the carbodiimide obtained in the carbodiimidization is pelletized in unpurified form or after purification.

13. A method of inhibiting against hydrolytic decomposition in ester-based polymers, comprising incorporating a carbodiimide according to claim 1 into an ester-based polymer composition.

14. A method of protection against hydrolytic degradation in thermoplastic polyurethane (TPU) comprising incorporating a carbodiimide according to claim 1 into a thermoplastic polyurethane composition.

15. A composition containing at least one inventive carbodiimide according to claim 1 and at least one ester-based polymer.

16. The composition according to claim 15, wherein the ester-based polymer is thermoplastic polyurethane (TPU) or a blend of ester-based polymers.

17. The carbodiimide according to claim 1, wherein R^{I} is triisopropylphenyl.

18. The carbodiimide according to claim 1, wherein n is from 1 to 100.

19. The carbodiimide according to claim 1, wherein n is from 1 to 50.

20. The carbodiimide according to claim 1, wherein n is from 0 to 20.
